(19) United States
(12) Patent Application Publication
(10) Pub. No.: US 2025/0318914 A1
(43) Pub. Date: Oct. 16, 2025

(54) MAXILLOFACIAL DEVICE AND METHOD FOR PLACEMENT DURING A LEFORT I OSTEOTOMY

(71) Applicant: Pasquale Scutari, JR., Skaneateles, NY (US)

(72) Inventor: Pasquale Scutari, JR., Skaneateles, NY (US)

(21) Appl. No.: 19/080,048

(22) Filed: Mar. 14, 2025

Related U.S. Application Data
(60) Provisional application No. 63/632,210, filed on Apr. 10, 2024.

(51) Int. Cl. A61C 8/00; A61B 34/10

(52) U.S. Cl. CPC A61C 8/0092; A61B 34/10; A61B 2034/102

(57) ABSTRACT

A maxillofacial device includes a substructure with a shape that is custom made to a patient's maxillofacial area; a framework coupled to the substructure, the framework and substructure being made of a single continuous material or a combination of materials, the framework having a plurality of grid elements; and one or more plate fixations coupled to the substructure. The framework has a shape that is custom made to fit the patient's maxillary sinus area. The framework is configured to be surrounded by a bone graft material, wherein the substructure and framework provide rigidity and stiffness for the bone graft material.

### CROSS-REFERENCE TO RELATED APPLICATIONS

This application claims the benefit of priority to U.S. Provisional application No. 63/632,210, filed on Apr. 10, 2024. That prior application is incorporated herein by reference in its entirety.

### BACKGROUND

A Lefort I Osteotomy (LeFort procedure) is a procedure commonly done on patients in order to reposition the maxilla to correct a dentofacial deformity. This procedure may involve a down fracture of the upper jaw and placing medical grade hardware in the patient. The procedure was named after the fracture pattern originally described by Rene LeFort in 1901 that extends from the nasal septum, along the tooth apices, and through the pterygomaxillary junction. See Buchanan E P, Hyman C H. LeFort I Osteotomy. Semin Plast Surg. 2013 August;27(3):149-54. doi: 10.1055/s-0033-1357112. PMID: 24872761; PMCID: PMC3805729 (incorporated herein by reference).

For patients in need of dental implants for edentulous maxillary atrophy, one option is place dental implants with the traditional intraoral approach, then to perform a Lefort I osteotomy and bone graft the sinus and/or nasal floor regions.

### SUMMARY

In some aspects, the techniques described herein relate to a maxillofacial device including: a substructure with a shape that is conformed, e.g., custom made, to a patient's maxillofacial area; a framework coupled to the substructure, the framework and substructure being made of a single continuous material or a combination of materials, the framework having a plurality of grid elements, wherein the framework has a shape that is conformed, e.g., custom made, to fit the patient's maxillary sinus area; and one or more plate fixations coupled to the substructure; wherein the framework is configured to be surrounded by a bone graft material, wherein the substructure and framework provide rigidity and stiffness for the bone graft material.

In some aspects, the techniques described herein relate to a method of installing a maxillofacial device, the method including: scanning a patient's maxillofacial area to obtain a computer model for use in manufacturing a maxillofacial device; manufacturing the maxillofacial device using the computer model by milling or hybrid manufacturing a single continuous piece of material or combination of materials, the maxillofacial device containing a plurality of frameworks and a plate fixation; performing a Lefort I Osteotomy on the patient to create a space to place the maxillofacial device; placing the maxillofacial device; and screwing the maxillofacial device to the maxilla and facial bones of the patient.

In some aspects, the techniques described herein relate to a medical device including: a metal and/or nonmetal substructure and/or other rigid material, the substructure made of a single continuous or combination of material, wherein the substructure matches a scanned patient's maxilla geometry; a framework that is made of the single continuous material, and wherein the framework contains a plurality of openings; and a plurality of plate fixations, the plurality of plate fixations configured to fixate the metal structure and the framework to the patient's maxilla and facial bones.

### BRIEF DESCRIPTION OF THE DRAWINGS

FIG. 1 shows an upper or superior view of the maxillofacial device that a surgeon may see during installation.

FIG. 2 shows a lateral view of the maxillofacial device of FIG. 1.

FIG. 3 shows a lower or inferior view inside of the oral cavity.

FIG. 4 shows a frontal view of the jaw structure with related hardware.

FIG. 5 shows a frontal view of dental implant connections.

FIG. 6 shows a flowchart for the placement of the maxillofacial device.

FIG. 7A. is an inferior view of the substructure and implant connections.

FIG. 7B is a partial cross-sectional view of the embodiment of FIG. 7A.

FIG. 8 is a perspective view of an example of the maxillofacial device installed in an oral cavity.

The same reference numbers or other reference designators are used in the drawings to designate the same or similar (functionally and/or structurally) features.

### DETAILED DESCRIPTION

Some patients who have bone loss in their maxillary bone and missing teeth, may require a severe edentulous atrophy reconstruction. This procedure may involve a bone graft and multiple dental implants, and it is common for the bone graft and multiple implants to have a higher failure rate. The failure may be caused by, for example, insufficient bone volume and quality as well as lack of primary stability of the dental implants. Such failures can require multiple surgical procedures with limited success.

Accordingly, various examples of the present description are related to a maxillofacial device which comprises a substructure and a framework. The substructure may be disposed around the maxillary sinus and may extend flush along the nasal floor. The framework may be disposed under the maxillary sinus and/or nasal floor and provides an increase in rigidity and stiffness for a bone graft material to aid in osteointegration. The maxillofacial device may be attached to the maxilla, or the upper fixed bone of the facial region, by means of a plurality of plate fixation screws. (both interlocking and/or non-interlocking)

The maxillofacial device may be installed during a Lefort I Osteotomy type procedure. The Lefort I Osteotomy may include a down-fracture of the upper jaw to provide a region for the maxillofacial device to be installed. In an embodiment, the maxillofacial device may be made unique and based on an intraoral scan and 3D imaging of every patient, so that the maxillofacial device geometry may vary. The scan may be conducted by an intraoral scanner, 3D X-ray, an MRI, or other suitable medical imaging technology.

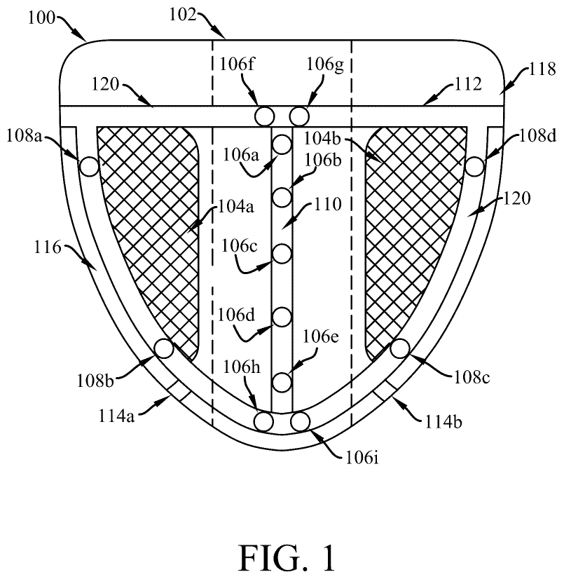

FIG. 1 shows an example of a superior, or upper, view of the maxillofacial device 100. This view shows the device in an installed location in a patient, wherein a nasal floor 102 extends posterior to anterior, a maxillary sinus 116 is disposed on both sides of the nasal floor 102, and the maxillofacial device 100 is inserted therein.

In this embodiment, the maxillofacial device 100 has a substructure 112 and a framework 104a-104b. The substructure 112 device has an outer corner region 118, a middle region 110, and an outer circumferential region 120. There are locking and/or non-locking fixation screws 106a-106i along the middle region 110, a plurality of dental implant components 108a-108d along the outer circumferential region 120, and extended regions 114a-114b.

In this embodiment, the middle region 110 of the substructure 112 is configured to be flush against the nasal floor 102. The framework 104a-104b has a curved shape to cup around the maxillary sinus 116. The framework 104a-104b is constructed with a rigid material such as titanium. The framework 104a-104b may also be constructed with resorbable material. In an embodiment, the framework 104a-104b has a plurality of grid elements, and each grid element is in the shape of a rectangle. Other grid elements shapes may also be used such as a triangle, hexagon, or other suitable polygon. The grid elements may also be circular. The framework 104a-104b may also plane or contoured surface with or without protrusions and/or holes.

The material used for the framework and/or the substructure can be the same or different. In an embodiment, the material in either case is rigid. By rigid it is meant a material having an ultimate tensile strength of 240 MPa to 1764 MPa, such as, for example, 300 to 1400 MPa, 500 to 1200 MPa, or 800 to 1050 MPa. In an embodiment, the device may have different thicknesses or types of material in the plate fixations, framework, and substructure. For example, in an embodiment, the plate fixations to attach to facial bones will be more malleable and less rigid than the framework and substructure, either by having a different thickness or a different type of material. Ultimate tensile strength can be determined by R. A. Loukota and J. C. Shelton, “Mechanical Analysis of Maxillofacial Miniplates,” Brit. J. of Oral and Maxillofacial Surgery, 33, 174-179 (1995), incorporated herein by reference.

In an embodiment, the framework 104a-104b may have bone graft material placed inside, so that the framework 104a-104b is encased in the bone graft material that eventually becomes contiguous to the bone of the patient.

One or more components of the maxillofacial device 100 may be made of, for example, medical grade 5 titanium or other suitable metal, metal alloy, and/or another material with similar rigidity. The bone graft material may be Autograft, allograft, xenograft, synthetic and/or an organic material.

The locking and/or non-locking fixation screws 106a-106i may be used to securely attach the maxillofacial device 100 to the maxilla bone of the patient. While multiple locking fixation screws 106a-106i are shown in the Figure, in other embodiments, there may be more or less locking fixation screws according to individual patient geometry. In an embodiment, the device 100 includes 4 to 20, screws, such as, for example, 6 to 16, or 8 to 12 screws.

The dental implant components 108a-108d are placed into the maxilla to provide a region for the dental implant and/or prosthesis to be attached to. It is noted that although only four extended regions 114a-114b are shown, more than four may be implemented. In an embodiment, the number of extended regions may be 2 to 10 or 3 to 6.

The maxillofacial device 100 is a single continuous material or a combination of materials between the framework 104a-104b and the substructure 112, so that the framework 104a-104b is stiff.

The stiffness of the maxillofacial device 100 is believed to aid in the healing process upon completion of the surgery. This is because the stiffness of the framework 104a-104b along with the rigidity of the substructure 112 provide a stable portion for the bone graft material to osteointegrate into. The locking fixation screws 106a-106i aid in this stiffness by securely attaching the maxillofacial device 100 to the maxilla. The locking fixation screws 106a-106i may also osteointegrate into the maxilla.

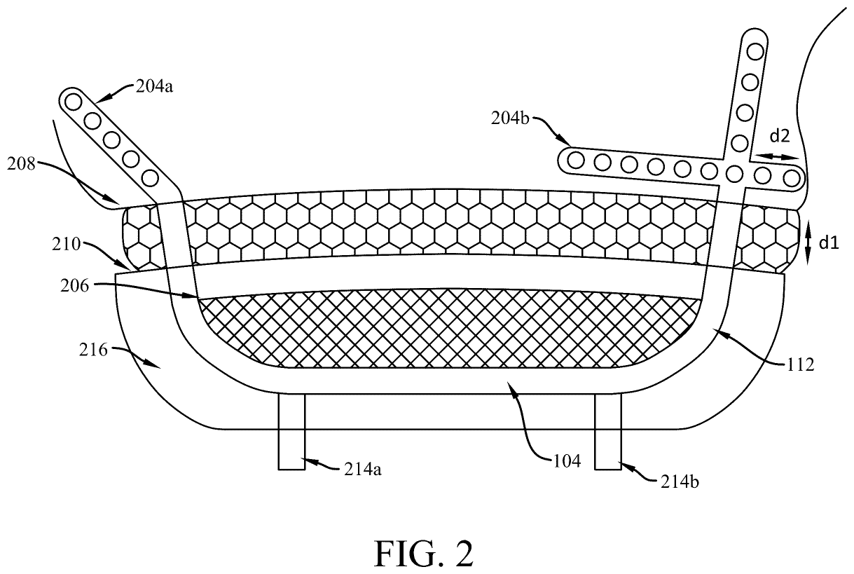

FIG. 2 shows a lateral view of the maxillofacial device 100 in a patient. The lateral view direction for FIG. 2 may be seen from the view direction as shown in FIG. 1. FIG. 2 shows the substructure 112, the framework 104, the dental implants 214a-214b, the plate fixations 204a-204b, top of the framework 206, a first cut line 208, a second cut line 210, and residual bone 216.

The plate fixations 204a-204b attach the maxillofacial device 100 to the maxilla. The bone graft (shown by the circles) is placed above the framework 104 and within the framework 104. The vertical distance, d1, and the anterior posterior distance, d2 may vary depending on patient geometry. The residual bone 216 surrounds the substructure 112 and the dental implants 214a-214b.

The plate fixations 204a-204b attach to the maxillofacial device 100 such that the maxillofacial device 100 is screwed into the maxilla. The maxillofacial device 100 provides a stiff structure for the bone graft material to osteointegrate into. Osetointegration is enhanced by the framework 104 of the maxillofacial device 100, which has cavities that the bone graft can fit into. The maxillofacial device also provides dental implant connections 214a-214b that dental implants, e.g., dentures, teeth, or crowns, can attach to. The plate fixations 204a-204b may be a malleable material. The plate fixations 204a-204b may also be hinged or unhinged, so that a portion of the plate fixations 204a-204b can be bent into the small location of the patient's oral cavity prepared for it.

Because patient geometry can vary widely, there may be a vertical distance formed by an upright component of the substructure 112, d1, comprising excess bone graft material that is not within the framework 104. The distance, d1, may, for example, be −10 to 50 mm, such as, −3 to 35 mm, or −10 to 20 mm. The distance, d2, may, for example, be −10 to 50 mm, such as −3 to 35 mm, or −10 to 20 mm.

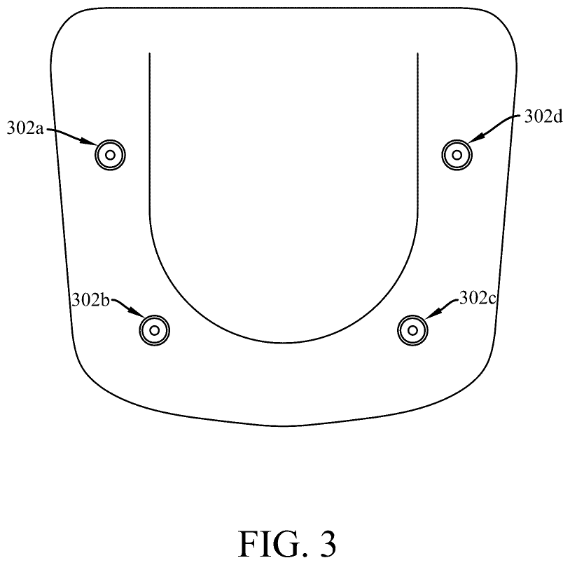

FIG. 3 shows an inferior, or bottom, view of the dental implant connections 302a-302d. The implant connections 302a-302d correspond to the bottom of the dental implant components or connection. 108a-108d in FIG. 1. These implant connections 302a-302d may provide a structure for an all-on-X prothesis. The shown example is all-on-4. Thus, allowing the maxillofacial device 100 to provide both: a rigid structure and an all-on-X prothesis connection.

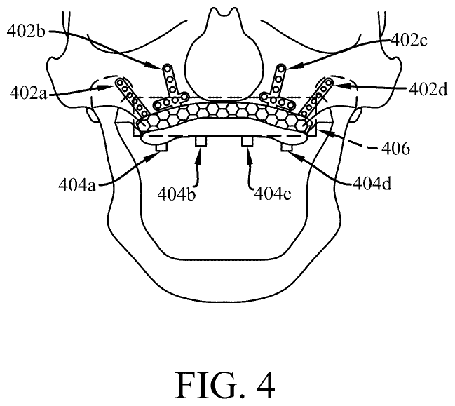

FIG. 4 shows a frontal view with plate fixations 402a-402d, dental implant connections 404a-404d, and a region 406 where the maxillofacial device (not shown) is to be placed.

The plate fixations 402a-402d extend through the maxilla bone structure to secure the maxillofacial device 100 to the maxilla and facial bones. The dental implant connections 404a-404d provide a region for dental implants to be connected to. The maxillofacial device 100 contains the plate fixations 402a-402d, so that the maxillofacial device 100 and plate fixations 402a-402d are made of a single continuous piece of material or a combination of materials. The maxillofacial device provides rigidity for a bone graft material to grow into, such that the maxillofacial device osteointegrates with bone graft material.

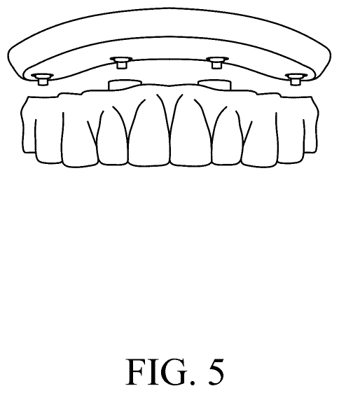

FIG. 5 shows an example on how dental implant connections are used to provide a prosthesis.

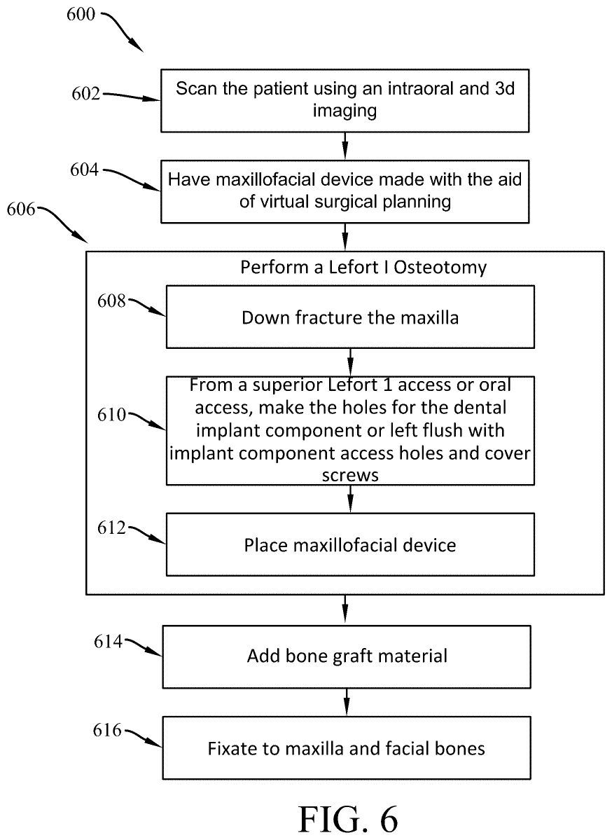

FIG. 6 shows a method 600 for placing the maxillofacial device.

At step 602 the patient's is scanned using an intraoral scanner and 3d imaging then they are merged together to create an accurate 3D representation of the patient's relevant oral geometry. The scanning may be done by a technician using a handheld device or some other suitable scanning technique. The scanning may be an intraoral scanner, 3D X-ray, an MRI, or other suitable medical scanning technology. The scan may produce a computer model file, for example .stl files, of the patient's maxillofacial geometry.

At step 604 the maxillofacial device is made with the aid of virtual surgical planning provided by software. The maxillofacial device may be milled, hybrid manufactured, and/or made by other means such as 3D printing from a single continuous piece or combination of materials or other suitable manufacturing methods. Hybrid manufacturing is the combination of additive and subtractive manufacturing in a single machine, e.g., a directed energy deposition head for depositing metal powder or wire combined with a machine cutting tools such as a lathe or mill. The maxillofacial device may be made of grade 5 medical titanium, or other suitable material. The device may be made to conform around the patient's geometry based on the computer file that was produced during the scan.

At step 606 a Lefort I Osteotomy is performed. The Lefort I Osteotomy may include steps 608-612. The Lefort I Osteotomy may include a team of medical professionals including, for example, a maxillofacial surgeon.

At step 608 a down fracture of the maxilla is performed. The down fracture may involve making one or more cuts into the maxilla bone through the sinus or nasal cavity. The down fracture provides a region for the maxillofacial device to be placed.

At step 610 holes are made for the maxillofacial device to be attached, e.g., at the plate fixations and the substructure. The holes may be made by means of a custom osteotomy jig that is also created based on the scans. The holes may be made by drilling into the maxilla bone from the superior or oral approach. The number of holes and the size of the holes may be dependent upon patient geometry. The device may be left flush with the maxilla on the superior aspect with the implant component access site protected with a cover screw.

At step 612 the maxillofacial device is placed. The maxillofacial device may slide into a proper position by means of another jig, e.g., a jig used for a Lefort I Osteotomy. The jig may be separately made and custom to the patient based on the scan. The maxillofacial device contains a framework that provides a region for the bone graft material to osteointegrate into. The maxillofacial device also provides stiffness and rigidity to aid in healing.

At step 614 bone graft material is added into the region created from the down fracture. The bone graft material may be autograft, allograft, xenograft, synthetic and/or organic material.

At step 616, the maxillofacial device is fixated to the maxilla and facial bones, e.g., with screws. The screws extend through the maxillofacial device and into the holes. The arrangement of dental implants, screws, maxillofacial device, and bone graft material provide a flush and stiff connection to the maxilla and facial bones.

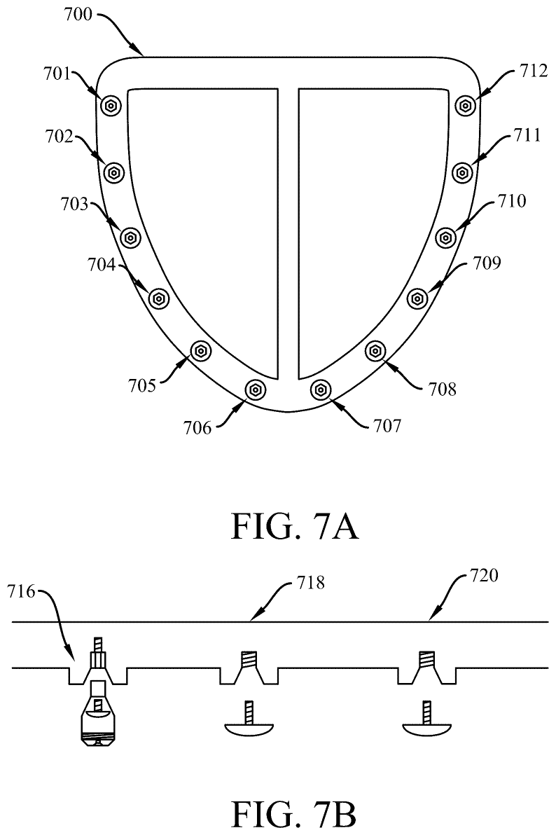

FIG. 7A shows an inferior view of the maxillofacial device. This view is consistent with placement of the device against the maxillary sinus/nasal floor. In this view, a plurality of implant ports 701-712 are shown. The implant points 701-712 may have a cover screws within them.

FIG. 7B further shows the embodiment of FIG. 7A in a partial cross sectional view. Three embodiments of attachments of the maxillofacial device 716-720 to the implant ports (e.g., 701-712) are shown with a Morse Taper and hex connection. In use, the attachments of the maxillofacial device will protrude through the roof of the mouth, typically through the alveolar gum line. FIG. 5 can be referred to as showing this. The first embodiment 716 shows a cover screw disposed within the hole by an abutment. The abutment may be a single-unit abutment or a multi-unit abutment similar to any currently available abutment options. The second embodiment 718 and the third embodiment 720 both show examples of a cover screw placed within the device as a temporary means to protect the device and not irritate the patient. For example, a cover screw may be placed within the device for a period of two months, then afterwards the cover screw can be removed and a cover screw to fit with a maxillofacial device implant extension is attached.

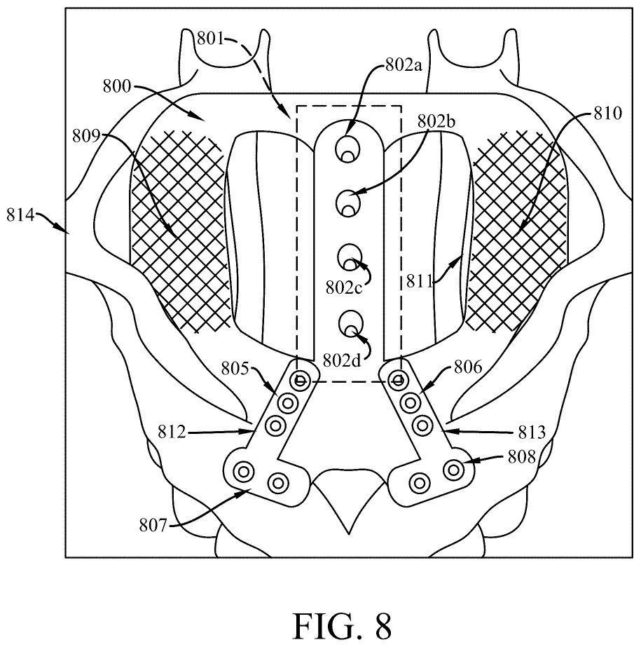

FIG. 8 shows an example of a maxillofacial device 800 positioned over an inferior segment 814 of the upper jaw, e.g., after a down fracture has been performed on a patient. The maxillofacial device 800 includes a center region 801 and an outer ring 811. The center region 801 includes multiple holes 802a-802d. The outer ring 811 includes a first mesh structure 809 and a second mesh structure 810. The maxillofacial device further includes a bone plate 812 and a second bone plate 813. In an embodiment there may be multiple areas with plate and screw fixation. The maxillofacial device includes first and second holes 805, 806 that are positioned adjacent to the first and second bone plates 812, 813. The first and second bone plates 812, 813 also includes third and fourth holes 807, 808 that extend in a direction approximately perpendicular to first and second holes 805-806. Bone graft material is placed within and around the first and second mesh structures 809, 810.

The combination of the center region 801, outer ring 811, multiple holes 805-808, and first and second bone plates 812, 813 allow the maxillofacial device 800 to have a high level of rigidity while the patient heals. This rigidity advantageously stabilizes the bone graft material within the first and second mesh structures 809, 810.

The maxillofacial device 800 may be made of a rigid material, for example, titanium or other suitable material. While only four holes 802a-802d are shown throughout the center region, it is understood that more or less holes may be used. For example, there may be 2-10 holes, or 4-6 holes.

What has been described above includes examples of one or more embodiments. It is, of course, not possible to describe every conceivable modification and alteration of the above devices or methodologies for purposes of describing the aforementioned aspects, but one of ordinary skill in the art can recognize that many further modifications and permutations of various aspects are possible. Furthermore, to the extent that the term “includes” is used in either the detailed description or the claims, such term is intended to be inclusive in a manner similar to the term “comprising” as “comprising” is interpreted when employed as a transitional word in a claim. The term “consisting essentially” as used herein means the specified materials or steps and those that do not materially affect the basic and novel characteristics of the material or method. If not specified above, any properties or measurements mentioned herein may be determined by applicable ASTM standards, or if an ASTM standard does not exist for the property, the most commonly used standard known by those of skill in the art may be used. The articles “a,” “an,” and “the,” should be interpreted to mean “one or more” unless the context indicates the contrary.

The term “or” is intended to mean an inclusive “or” rather than an exclusive “or.” That is, unless specified otherwise, or clear from the context, the phrase “X employs A or B” is intended to mean any of the natural inclusive permutations. That is, the phrase “X employs A or B” is satisfied by any of the following instances: X employs A; X employs B; or X employs both A and B. In addition, the articles “a” and “an” as used in this application and the appended claims should generally be construed to mean “one or more” unless specified otherwise or clear from the context to be directed to a singular form. Additionally, as used herein, the term “exemplary” is intended to mean serving as an illustration or example of something, and is not intended to indicate a preference.

What is claimed is:

1. A maxillofacial device comprising:
a substructure with a shape that is conformed to a maxillofacial area of a patient;
a framework coupled to the substructure, the framework and substructure being made of a single continuous material or a combination of materials, the framework having a plurality of grid elements, wherein the framework has a shape that is conformed to fit the maxillary sinus area of the patient; and
one or more plate fixations coupled to the substructure;
wherein the framework is configured to be surrounded by a bone graft material, wherein the substructure and framework provide rigidity and stiffness for the bone graft material.

2. The maxillofacial device of claim 1, wherein the single continuous or combination of materials comprises titanium and/or other suitable material.

3. The maxillofacial device of claim 1, wherein the bone graft material is autograft, allograft, xenograft, synthetic, or a combination of any of these.

4. The maxillofacial device of claim 1, wherein the shape is determined by means of an intraoral scan and 3D imaging and/or any more advanced technology.

5. The maxillofacial device of claim 1, wherein the substructure further has a middle region that is configured to lie flush against a patient's nasal floor, wherein the middle region of the substructure is configured to receive one or more locking fixation screws.

6. The maxillofacial device of claim 1, wherein the framework is disposed along both sides of a middle region of the substructure, and wherein the framework is disposed below a maxillary sinus region of the patient.

7. The maxillofacial device of claim 6, wherein the substructure is configured to receive dental implants and/or function as dental implants.

8. The maxillofacial device of claim 7, wherein the substructure framework are rigid.

9. A method of installing a maxillofacial device, the method comprising:
scanning a patient's maxillofacial area to obtain a computer model for use in manufacturing a maxillofacial device;
manufacturing the maxillofacial device using the computer model by milling or hybrid manufacturing a single continuous piece of material or combination of materials, the maxillofacial device containing a plurality of frameworks and a plate fixation;
performing a Lefort I Osteotomy on the patient to create a space to place the maxillofacial device;
placing the maxillofacial device; and
screwing the maxillofacial device to the maxilla and facial bones of the patient.

10. The method of claim 9, wherein the maxillofacial device comprises titanium and/or another suitable material.

11. The method of claim 9, further comprising:
placing bone graft material in or around the maxillofacial device.

12. The method of claim 11, wherein the bone graft material is from another bone of the patient.

13. The method of claim 11, wherein the bone graft material is xenograft, allograft, synthetic, organic and or a combination of these.

14. The method of claim 9, wherein the scanning of the patient is with an intraoral scan and 3D imaging.

15. The method of claim 9, wherein the Lefort I Osteotomy comprises a down fracture, and wherein the maxillofacial device is placed in a region created from the down fracture.

16. A medical device comprising:
a metal and/or nonmetal substructure and/or other rigid material, the substructure made of a single continuous or combination of material, wherein the substructure matches a scanned patient's maxilla geometry;
a framework that is made of the single continuous material, and wherein the framework contains a plurality of openings; and
a plurality of plate fixations, the plurality of plate fixations configured to fixate the metal structure and the framework to the patient's maxilla and facial bones.

17. The medical device of claim 16, wherein the single continuous and/or combination comprises titanium and/or other rigid material.

18. The medical device of claim 16, wherein the plurality of plate fixations are made of titanium alloy and/or other rigid material.

19. The medical device of claim 16, wherein the framework is configured to provide a region for a bone graft to osteointegrate into.

20. The medical device of claim 16, wherein the metal substructure has holes, wherein the plurality of plate fixations are configured to be coupled to the substructure with fasteners extending through the holes.

21. The maxillofacial device of claim 1, wherein the bone graft material is organic.
